(19) United States
(12) Patent Application Publication
(10) Pub. No.: US 2025/0230119 A1
(43) Pub. Date: Jul. 17, 2025

(54) PROCESSES FOR PRODUCING ALKYL ACRYLATE DIMERS

(71) Applicant: SPECIALTY OPERATIONS FRANCE, Lyon (FR)

(72) Inventor: Olivier Back, Lyon (FR)

(73) Assignee: SPECIALTY OPERATIONS FRANCE, Lyon (FR)

(21) Appl. No.: 18/702,757

(22) PCT Filed: Oct. 17, 2022
(86) PCT No.: PCT/EP2022/078762

§ 371 Date: Apr. 18, 2024

(30) Foreign Application Priority Data
Oct. 18, 2021 (EP) 21203092.8

(51) Int. Cl. C07C 57/13; C07C 67/465

(52) U.S. Cl. CPC C07C 57/13; C07C 67/465

(57) ABSTRACT

The present invention relates to a process for producing an alkyl acrylate dimer. Furthermore, the present invention relates to a process for producing a hydrogenated alkyl acrylate dimer obtained by the dimerization process according to the present invention. Moreover, the present invention relates to a process of producing a hydrolyzed alkyl acrylate dimer obtained by the dimerization process according to the present invention.

### FIELD OF THE INVENTION

The present invention relates to a process for producing an alkyl acrylate dimer. Furthermore, the present invention relates to a process for producing a hydrogenated alkyl acrylate dimer obtained by the dimerization process according to the present invention. Moreover, the present invention relates to a process of producing a hydrolyzed alkyl acrylate dimer obtained by the dimerization process according to the present invention.

### TECHNICAL BACKGROUND

The use of specific phosphines as catalysts for the dimerization of alkyl acrylates via the Rauhut-Currier reaction has already been described in the prior art.

U.S. Pat. No. 3,074,999 A describes an alkyl acrylate dimerization reaction catalyzed by tertiary phosphines having three alkyl groups, three alicyclic groups or three aryl groups such as tributylphosphine or triphenylphosphine. However, these catalysts exhibit a low activity in the dimerization reaction. Regarding the disclosed process, moderate yields are reported which are severe drawbacks for a commercial production.

U.S. Pat. No. 3,227,745 A describes an alkyl acrylate dimerization reaction catalyzed by a tertiary phosphine in the presence of large amounts of tert-butyl alcohol as a solvent. The disclosed tertiary phosphines are trialkylphosphines. However, with the described process only a low conversion of the acrylate of less than 50% is achieved, which is not suitable for industrial production processes.

U.S. Pat. No. 3,342,853 A describes the acrylate dimerization catalyzed by triaminophosphines that can be generated prior to the dimerization reaction from PCl₃. Yields of 70-80% of the methyleneglutarate ester dimers are reported when the reaction is conducted at 60-65° C., however significant amounts of by-product is also generated. Furthermore, triaminophosphines are generally toxic and CMR reagents (carcinogenic, mutagenic and reprotoxic reagents) and when the catalyst is generated in-situ, PCl₃ is used as the precursor which is a very hazardous chemical. These are serious drawbacks for the commercialization and industrialization of this process.

U.S. Pat. No. 3,342,854 A describes acrylate dimerization reactions catalyzed either by mono-aminophosphines or bis-aminophosphines. However, the low activity of diphenylaminophosphines for the acrylate dimerization requires the use of high phosphine loadings which is a severe drawback for commercial productions. This is shown by two examples in this patent application using either an in-situ generated dibutylaminodiphenylphosphine catalyst or a diethylaminodiphenylphosphine catalyst which result to dimer yields equal to or less than 10%. Furthermore, using the process according to U.S. Pat. No. 3,342,854 A a significant amount of by-product is obtained.

Weiping Su et al. describe in “P(RNCH₂CH₂)₃N: Catalysts for the Head-to-Tail Dimerization of Methyl Acrylate” J. Org. Chem. 2003, 68, 9499-9501 methyl acrylate dimerization in THF or dioxane as solvents at room temperature using proazaphosphatranes as phosphine catalysts. At 1 mol % catalyst loading, up to 82% yield is obtained. However, the catalysts described in this paper are quite complex and difficult to synthetize resulting to overall expensive catalysts costs which is a severe drawback for a potential industrialization. Moreover, using low catalyst loadings (1 mol %), the reaction kinetics are slow at room temperature resulting to long reaction times (up to 24 h) which is also a drawback for industrial production.

### SUMMARY OF THE INVENTION

It is a problem of the present invention to provide an efficient process for producing an alkyl acrylate dimer, using a highly active, robust, reusable, inexpensive and readily accessible catalyst, wherein the catalyst is relatively low in toxicity, can be used with relatively low catalyst loadings and provides excellent selectivity.

Specifically, it is a problem of the present invention to provide a process for preparing an alkyl acrylate dimer, wherein the use of large amounts of tertiary alcohols as solvents and relatively high catalysts loadings can be avoided. More specifically, it is a problem of the present invention to provide an efficient process for preparing a hydrogenated alkyl acrylate dimer and an efficient process for preparing a hydrolyzed alkyl acrylate dimer.

It has now been found that these and other problems can be solved by the process of the present invention. The present invention relates to a process for producing a dimer according to formula (II) comprising a step i) of dimerization of alkyl acrylates according to formula (I) to obtain a dimer according to formula (II) using a catalyst according to formula (III), according to the following reaction scheme:

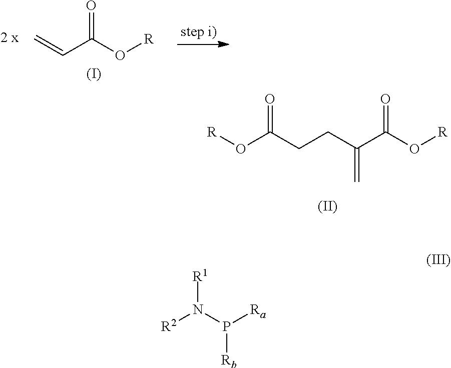

wherein
R is an alkyl group;
R₁ and R₂ which are identical or different, are either aliphatic groups or form together with the N atom a heteroaliphatic cycle;
Rₐ is a hydrocarbyl group;
R_{b} is either an aliphatic group or NR₃R₄ with R₃ and R₄ being identical or different, and being either aliphatic groups or forming together with the N atom a heteroaliphatic cycle;
and wherein said step i) of dimerization is performed in presence of a compound A being a tertiary alcohol or a silanol.

Furthermore, the present invention relates to a process as defined above, further comprising an initial step 0) of preparation of the catalyst according to formula (III) by reacting a compound according to formula (IV)

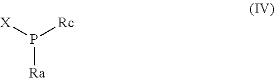

wherein
X is a chloride, a bromide or an iodide, preferably chloride;
Rₐ is as defined above;
R_{c} is either X (for the case of catalysts of formula (III) wherein R_{b} is NR₃R₄ as defined above) or R_{b} (for the case of catalysts of formula (III) wherein R_{b} is an aliphatic group);
with
   an amine of formula (V): R₁R₂NH (V), with R₁ and R₂ being as defined above when R_{c} is R_{b} or
   both an amine of formula (V) and an amine of formula (V′): R₃R₄NH (V′), with R₃ and R₄ being as defined above when R_{c} is X.

Moreover, the present invention provides a process for producing a compound according to formula (VI), comprising the process as defined above, followed by a step ii) of hydrogenation of the dimer according to formula (II) obtained in the step of dimerization using H₂ and a hydrogenation catalyst, such as Pd based catalysts, for example Pd/C, Pd/Al₂O₃, Pd/SiO₂, Ru based catalysts, for example Ru/C, Pt based catalysts such as Pt/C, Ni based catalysts such as supported nickel or Raney nickel catalysts, Co based catalyst such as supported cobalt or Raney cobalt, Rh based catalyst such as Rh/C, Ir based catalyst such as Ir/C, preferably Pd/C or Raney nickel, preferably Pd/C to obtain a compound according to formula (VI)

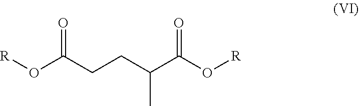

wherein R is as defined above.

Finally, the present invention relates to a process for producing a compound according to formula (VII), comprising the process as defined above, followed by a step ii′) of hydrolysis of the dimer according to formula (II) obtained in the step of dimerization using acid catalysts such as Lewis or Bronsted acids, for example: HCl, H₂SO₄, para-toluenesulfonic acid, methanesulfonic acid, triflic acid, solid acidic catalysts such as Amberlyst resins or zeolites, Nafion to obtain a compound according to formula (VII)

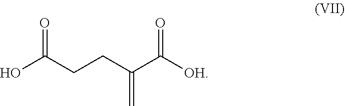

The present invention is based on the recognition that an efficient process for producing an alkyl acrylate dimer, using a highly active, robust, reusable, inexpensive and readily accessible catalyst is provided. The catalyst for the dimerization of alkyl acrylates is a compound according to formula (III) which is relatively low in toxicity, reusable, can be used at relatively low catalyst loadings and provides excellent selectivity. In addition, the present invention provides an efficient process for preparing an alkyl acrylate dimer using the compound according to formula (III) as a catalyst, wherein the use of large amounts of tertiary alcohols with respect to the alkyl acrylate and the use of relatively high catalyst loadings can be avoided. Specifically, the amount of the tertiary alcohol with respect to the alkyl acrylate can be reduced to a ratio of 0.01:1 and the catalyst loading can be reduced to 0.20 mol %. Finally, the present invention provides an efficient process for preparing a hydrogenated alkyl acrylate dimer and an efficient process for preparing a hydrolyzed alkyl acrylate dimer.

### DETAILED DESCRIPTION OF THE INVENTION

According to the present invention the term “about” means ±10% of the specified numeric value, preferably ±5% and most preferably ±2%.

The present invention relates to a process for producing a dimer according to formula (II) comprising a step i) of dimerization of alkyl acrylates according to formula (I) to obtain a dimer according to formula (II) using a catalyst according to formula (III), according to the following reaction scheme:

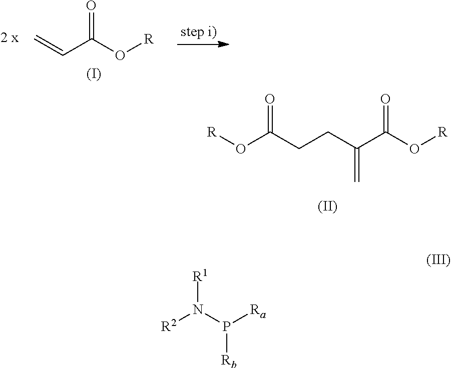

wherein
R is an alkyl group;
R₁ and R₂ which are identical or different, are either aliphatic groups or form together with the N atom a heteroaliphatic cycle;
Rₐ is a hydrocarbyl group;
R_{b} is either an aliphatic group or NR₃R₄ with R₃ and R₄ being identical or different, and being either aliphatic groups or forming together with the N atom a heteroaliphatic cycle;
and wherein said step i) of dimerization is performed in presence of a compound A being a tertiary alcohol or a silanol.

Preferably, in the process for producing a dimer according to formula (II) as defined herein, the compound A is a tertiary alcohol, such as tert-butanol, tert-amyl alcohol or pinacol and more preferably tert-butanol.

Preferably, in the process for producing a dimer according to formula (II) as defined herein, the molar ratio [compound A]/[alkyl acrylate according to formula (I)] is selected from about 4:1 to about 0.01:1, preferably from about 2:1 to about 0.1:1 and more preferably from about 0.5:1 to about 0.1:1, and notably from about 0.5:1 to about 0.2:1.

Preferably, in the process for producing a dimer according to formula (II) as defined herein, R is a C₁-C₁₈, more preferably a C₁-C₈ alkyl, still more preferably a C₁-C₄ alkyl and most preferably a methyl.

Preferably, in the process for producing a dimer according to formula (II) as defined herein, R is a methyl group, ethyl group, propyl group, isopropyl group, n-butyl group, sec-butyl group, isobutyl group, tert-butyl group, pentyl group, hexyl group, 2-ethylhexyl group, octyl group, decyl group, dodecyl group, t-dodecyl group, tetradecyl group, hexadecyl group or an octadecyl group, more preferably, a methyl group, ethyl group, isopropyl group, butyl group or 2-ethylhexyl group, still more preferably a methyl group, ethyl group, isopropyl group or a butyl group, most preferably a methyl group.

Preferably, in the process for producing a dimer according to formula (II) as defined herein, R₁ and R₂ are identical linear or branched alkyl groups comprising from 1 to 6 carbon atoms, more preferably from 1 to 3 carbon atoms, most preferably ethyl.

Preferably, in the process for producing a dimer according to formula (II) as defined herein, R₁ and R₂ form together with the N atom a heteroaliphatic cycle comprising from 3 to 5 carbon atoms, preferably 4 carbon atoms.

Preferably, in the process for producing a dimer according to formula (II) as defined herein, Rₐ is either an aromatic or an aliphatic group, more preferably an aromatic group, still more preferably selected from phenyl, tolyl, xylyl, mesityl, duryl, pentamethylphenyl, 2,6-diisopropylphenyl, tert-butylphenyl, ditert-butylphenyl, methoxyphenyl, dimethoxyphenyl, methoxytolyl, methylenedioxyphenyl, biphenyl, nitrophenyl, halogen substituted phenyl, trifluoromethylphenyl, naphtyl, pyridyl, furyl, pyrrolyl, thiophenyl, 2-indolyl, benzofuryl and all their position isomers.

Preferably, Rₐ is selected from phenyl; ortho-, meta- or para-tolyl; xylyl including all position isomers such as: 2,3-dimethylphenyl, 2,4-dimethylphenyl, 2,5-dimethylphenyl, 2,6-dimethylphenyl, 3,4-dimethylphenyl and 3,5-dimethylphenyl; 3-methyl-4-methoxyphenyl, 2-methyl-4-methoxyphenyl, 2-methyl-3-methoxyphenyl, 4-methyl-3-methoxyphenyl, 5-methyl-3-methoxyphenyl, 6-methyl-3-methoxyphenyl 2-methoxy-3-methylphenyl, 2-methoxy-4-methylphenyl, 2-methoxy-5-methylphenyl, 2-methoxy-6-methylphenyl; mesityl including all position isomers as: such 2,3,4-trimethylphenyl, 2,3,5-trimethylphenyl, 2,3,6-trimethylphenyl, 2,4,5-trimethylphenyl, 2,4,6-trimethylphenyl and 3,4,5-trimethylphenyl; duryl including all position isomers such as: 2,3,4,5-tetramethylphenyl, 2,3,4,6-tetramethylphenyl and 2,3,5,6-tetramethylphenyl; pentamethylphenyl, 2,6-diisopropylphenyl; ortho-, meta- or para-tert-butylphenyl; 2,3-di-tert-butylphenyl, 2,4-di-tert-butylphenyl, 2,5-di-tert-butylphenyl, 2,6-di-tert-butylphenyl, 3,4-di-tert-butylphenyl and 3,5-di-tert-butylphenyl; ortho-, meta- or para-methoxyphenyl; ortho-, meta- or para-chlorophenyl; 2,3-dimethoxyphenyl, 2,4-dimethoxyphenyl, 2,5-dimethoxyphenyl, 2,6-dimethoxyphenyl, 3,4-dimethoxyphenyl and 3,5-dimethoxyphenyl; 2,3-methylenedioxyphenyl, 3,4-methylenedioxyphenyl; ortho-, meta- or para-nitrophenyl; ortho-, meta- or para-biphenyl; ortho-, meta- or para-trifluoromethylphenyl, ortho-, meta- or para-fluorophenyl; 1- or 2-naphtyl; 2-pyridyl, 3-pyridyl or 4-pyridyl; 2-furyl, 3-furyl; 1-pyrrolyl, 2-pyrrolyl or 3-pyrrolyl; 2-thiophenyl, 3-thiophenyl; 2-indolyl, 3-indolyl, 2-benzofuryl and 3-benzofuryl; preferably phenyl; ortho-, meta- or para-tolyl; or xylyl and its isomer positions.

Preferably, in the process for producing a dimer according to formula (II) as defined herein, R_{b} is NR₃R₄ with R₃ and R₄ being identical or different, and being either an aliphatic group or forming together with the N atom a heteroaliphatic cycle, more preferably R₃ and R₄ are identical linear or branched alkyl groups comprising from 1 to 6 carbon atoms, still more preferably from 1 to 3 carbon atoms, most preferably ethyl.

Preferably, in the process for producing a dimer according to formula (II) as defined herein, Rₐ is a phenyl, R₁ and R₂ are ethyl and R_{b} is NR₃R₄ with R₃ and R₄ being ethyl.

Preferably, in the process for producing a dimer according to formula (II) as defined herein, the catalyst according to formula (III) is a compound selected from the group consisting of the compounds according to formulae (VIII) to (XIV):

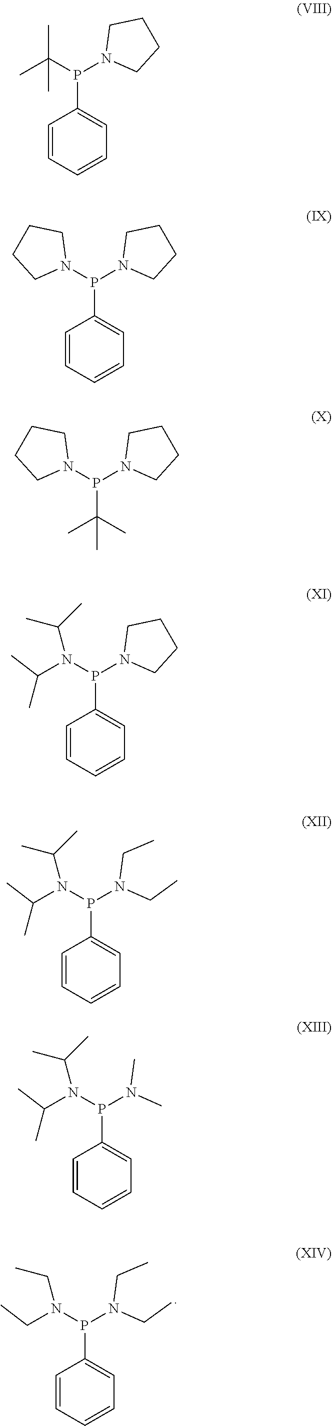

More preferably, in the process for producing a dimer according to formula (II) as defined herein, the catalyst according to formula (III) is a compound selected from the group consisting of compounds according to formulae (IX) and (XI) to (XIV), still more preferably selected from the group consisting of compounds according to formulae (XI), (XII) and (XIV), even more preferably selected from the group consisting of compounds according to formulae (XI) and (XIV), most preferably the catalyst according to formula (III) is the compound according to formula (XIV).

Preferably, in the process for producing a dimer according to formula (II) as defined herein, the step i) of dimerization is performed in an organic solvent, more preferably an aprotic solvent, still more preferably selected from tetrahydrofuran (THF), methyl-tetrahydrofuran (MeTHF), toluene, xylene, anisole, diethyl ether, tert-butyl methyl ether (MTBE), dichloromethane (DCM), chloroform, dioxane, pentane, cyclopentane, hexane, cyclohexane, methylcyclohexane, benzene and acetonitrile, even more preferably MeTHF, anisole and toluene, most preferably MeTHF and anisole.

Preferably, in the process for producing a dimer according to formula (II) as defined herein, the dimerization step i) is performed at a temperature ranging from about 20° C. to about 120° C., more preferably about 20° C. to about 80° C., still more preferably about 25° C. to about 60° C., most preferably from about 30° C. to about 60° C.

Preferably, in the process for producing a dimer according to formula (II) as defined herein, in step i) the catalyst according to formula (III) is used with a catalyst loading of from 0.20 mol % to 1.00 mol % with respect to the alkyl acrylate according to formula (I), more preferably of from about 0.25 mol % to about 0.90 mol %, still more preferably of from about 0.30 mol % to about 0.90 mol %, even more preferably of from about 0.30 mol % to about 0.80 mol %, even still more preferably of from about 0.30 mol % to about 0.70 mol %, even still more preferably of from about 0.30 mol % to about 0.60 mol %, most preferably of from about 0.30 mol % to 0.50 mol %.

Preferably, in the process for producing a dimer according to formula (II) as defined herein, the dimerization step i) is carried out in anhydrous conditions and in the absence of oxygen.

Preferably, the process for producing a dimer according to formula (II) as defined herein, further comprises an initial step 0) of preparation of the catalyst according to formula (III) by reacting a compound according to formula (IV)

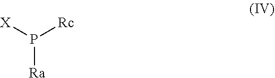

wherein
X is a chloride, a bromide or an iodide, preferably chloride;
Rₐ is a hydrocarbyl group;
R_{c} is either X (for the case of catalysts of formula (III) wherein R_{b} is NR₃R₄ as defined above) or R_{b} (for the case of catalysts of formula (III) wherein R_{b} is an aliphatic group);
with
   an amine of formula (V): R₁R₂NH (V), with R₁ and R₂ being as defined herein when R_{c} is R_{b} or
   both an amine of formula (V) and an amine of formula (V′): R₃R₄NH (V′), with R₃ and R₄ being as defined herein when R_{c} is X.

Preferably, in the process for producing a dimer according to formula (II) as defined herein, step 0) and step i) are consecutive steps performed without isolation of the catalyst after step 0).

Preferably, in the process for producing a dimer according to formula (II) as defined herein, R_{c} is X.

Preferably, in the process for producing a dimer according to formula (II) as defined herein, the step 0) is performed in an organic solvent, more preferably an aprotic solvent, still more preferably selected from tetrahydrofuran (THF), methyl-tetrahydrofuran (MeTHF), toluene, xylene, anisole, diethyl ether, tert-butyl methyl ether (MTBE), dichloromethane (DCM), chloroform, dioxane, pentane, cyclopentane, hexane, cyclohexane, methylcyclohexane, benzene and acetonitrile, even more preferably MeTHF, anisole and toluene, most preferably MeTHF and anisole.

Preferably, in the process for producing a dimer according to formula (II) as defined herein, step 0) is performed at a temperature ranging from about 20° C. to about 100° C., preferably about 20° C. to 80° C., more preferably about 25° C. to 60° C., most preferably at a temperature of about 40° C.

Preferably, in the process for producing a dimer according to formula (II) as defined herein, step 0) is performed through the slow addition of the reactant of formula (IV) to a solution of the amine R₁R₂NH in the aprotic solvent where the amine is used in an amount equal to or greater than 2 equivalents with respect to the reactant of formula (IV) when R_{c} in (IV) is R_{b} and R_{b} is an aliphatic group. Step (0) can also be performed through the slow addition of the reactant of formula (IV) to a solution containing both the amine R₁R₂NH and R₃R₄NH in the aprotic solvent where the total amount of the amines is equal or greater than 4 equivalents with respect to the reactant for formula (IV) when Rc in (IV) is X.

Preferably, in the process for producing a dimer according to formula (II) as defined herein, step 0) is carried out in anhydrous conditions and in the absence of oxygen.

Preferably, in the process for producing a dimer according to formula (II) as defined herein, step 0) comprises a filtration step to remove ammonium chloride by-products formed before performing step i).

Moreover, the present invention provides a process for producing a compound according to formula (VI), comprising the process for producing a dimer according to formula (II) as defined herein, followed by a step ii) of hydrogenation of the dimer according to formula (II) obtained in the step of dimerization using H₂ and a hydrogenation catalyst, such as Pd based catalysts, for example Pd/C, Pd/Al₂O₃, Pd/SiO₂, Ru based catalysts, for example Ru/C, Pt based catalysts such as Pt/C, Ni based catalysts, such as supported nickel or Raney nickel catalysts, Co based catalyst, such as supported cobalt or Raney cobalt, Rh based catalyst, such as Rh/C, Ir based catalyst, such as Ir/C, preferably Pd/C or Raney nickel, preferably Pd/C, to obtain a compound according to formula (VI)

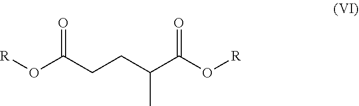

wherein R is as defined herein.

In a preferred embodiment, the process for producing a compound according to formula (VI) further comprises a step ii′) of hydrolysis of the hydrogenated dimer according to formula (VI) obtained in the step ii) using acid catalysts, such as Lewis or Bronsted acids, for example: HCl, H₂SO₄, para-toluenesulfonic acid, methanesulfonic acid, triflic acid, solid acidic catalysts, such as Amberlyst resins, zeolites or Nafion, to obtain a compound according to formula (XV).

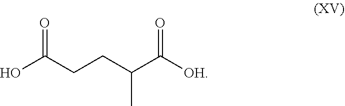

Finally, the present invention relates to a process for producing a compound according to formula (VII), comprising the process as defined above, followed by a step ii′) of hydrolysis of the dimer according to formula (II) obtained in the step of dimerization using acid catalysts such as Lewis or Bronsted acids, for example: HCl, H₂SO₄, para-toluenesulfonic acid, methanesulfonic acid, triflic acid, solid acidic catalysts such as Amberlyst resins or zeolites, Nafion to obtain a compound according to formula (VII).

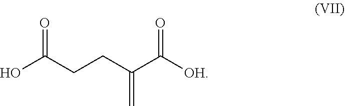

In a preferred embodiment, the process for producing a compound according to formula (VII) further comprises a step ii) of hydrogenation of the hydrolyzed dimer according to formula (VII) obtained in the step ii′) using H₂ and a hydrogenation catalyst, such as Pd based catalysts, for example Pd/C, Pd/Al₂O₃, Pd/SiO₂, Ru based catalysts, for example Ru/C, Pt based catalysts, such as Pt/C, Ni based catalysts, such as supported nickel or Raney nickel catalysts, Co based catalyst, such as supported cobalt or Raney cobalt, Rh based catalyst, such as Rh/C, Ir based catalyst such as Ir/C, preferably Pd/C or Raney nickel, preferably Pd/C, to obtain a compound according to formula (XV).

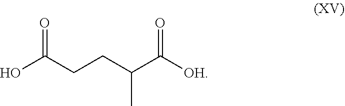

### Examples

### 1. Aminophosphine Catalyzed Methyl Acrylate Dimerization

### Phosphines Screening Study General Protocols:

a) Dimerization Catalyzed by Symmetrical Bis-Aminophosphines from Dichlorophosphines:

All the reactions were conducted in carefully dried vessels under an inert argon atmosphere. Methyl acrylate and tert-butanol were dried using 4 A molecular sieves and tert-butanol was distilled under argon prior to each reaction. Dichlorophosphines and amines were used as such.

In a 25 mL two necked round bottom flask are added:
3 mL of 2-methyltetrahydrofuran
dichlorophosphine precursor (1.8 mmoles, 0.01 eq. with respect to methyl acrylate).

In a 50 mL three necked round bottom flask equipped with a magnetic stirring device were added:
1 mL of 2-methyltetrahydrofuran
4 equivalents with respect to the dichlorophosphine precursor of the desired amine (7.2 mmoles).

The dichlorophosphine solution was progressively added to the amine solution under stirring (1400 rpm) over 1 hour while keeping the temperature of the reaction medium below 40° C. (exothermic reaction). Upon dichlorophosphine addition to the amine solution, there was formation of a white precipitate corresponding to the insoluble ammonium chloride salt by-product. At the end of the addition, the mixture was then allowed to stir at ambient temperature and the reaction progress was monitored thanks to ³¹P NMR (see table 1 below for the results of ³¹P chemical shifts of investigated aminophosphines).

After phosphine formation completion (which usually requires 1 h of stirring at room temperature after chlorophosphine addition for the unhindered amines and 2 h for the more sterically hindered amines) the mixture was then filtered via cannula into a 100 mL three necked round bottom flask equipped with a magnetic stirrer, a condenser, a heater and a temperature probe and containing 32 mL of melted tert-butanol (2:1 v/v with respect to methyl acrylate). The mixture was then allowed to stir at 60° C. Immediately 15.95 mL of methyl acrylate (15.15 g, 0.176 mole, 1 eq.) was carefully added over 1 hour (exothermy) into the reactor and the reaction progress was monitored thanks to ¹H NMR. Reactions were pursued until advancement ceased or until 1 day of reaction. The conversion of methyl acrylate was then estimated by ¹H NMR thanks to the integration of the methylene protons of the products and the methylene protons in the starting methyl acrylate.

NMR spectrum of the product:

¹H NMR (CDCl₃, 400 MHz) δ (ppm): 6.03 (s, 1H), 5.46 (s, 1H), 3.60 (s, 3H), 3.51 (s, 3H), 2.48 (t, J=7.6 Hz, 2H), 2.37 (t, J=7.6 Hz, 2H).

b) Dimerization Catalyzed by Mono-Aminophosphines from Mono-Chlorophosphines:

All the reactions were conducted in carefully dried vessels under an inert argon atmosphere. Methyl acrylate and tert-butanol were dried using 4 A molecular sieves and tert-butanol was distilled under argon prior to each reaction. Mono-chlorophosphines and amines were used as such.

In a 25 mL two necked round bottom flask were added:
3 mL of 2-methyltetrahydrofuran
Mono-chlorophosphine precursor (1.8 mmoles, 0.01 eq. with respect to methyl acrylate).

In a 50 mL three necked round bottom flask equipped with a magnetic stirring device were added:
1 mL of 2-methyltetrahydrofuran
2 equivalents with respect to the mono-chlorophosphine precursor of the desired amine (3.6 mmoles).

The mono-chlorophosphine solution was progressively added to the amine solution under stirring (1400 rpm) over 1 hour while keeping the temperature of the reaction medium below 40° C. (exothermic reaction). Upon mono-chlorophosphine addition to the amine solution, there was formation of a white precipitate corresponding to the insoluble ammonium chloride salt by-product. At the end of the addition, the mixture was then allowed to stir at ambient temperature and the reaction progress was monitored thanks to ³¹P NMR.

After phosphine formation completion (which usually requires 1 h of stirring at room temperature after mono-chlorophosphine addition for the unhindered amines and 2 h for the more sterically hindered amines) the mixture was then filtered via cannula into a 100 mL three necked round bottom flask equipped with a magnetic stirrer, a condenser, a heater and a temperature probe and containing 32 mL of melted tert-butanol (2:1 v/v with respect to methyl acrylate). The mixture was then allowed to stir at 60° C. Immediately, 15.95 mL of methyl acrylate (15.15 g, 0.176 mole, 1 eq.) was carefully added over 1 hour (exothermy) into the reactor and the reaction progress was monitored thanks to ¹H NMR. Reactions were pursued until advancement ceased or until 1 day of reaction. The conversion of methyl acrylate was then estimated by ¹H NMR thanks to the integration of the methylene protons of the products and the methylene proton in the starting methyl acrylate.

c) Dimerization Catalyzed by Unsymmetrical Bis-Aminophosphines from Dichlorophosphines, Diisopropylamine and an Additional Amine:

All the reactions were conducted in carefully dried vessels under an inert argon atmosphere. Methyl acrylate and tert-butanol were dried using 4 A molecular sieves and tert-butanol was distilled under argon prior to each reaction. Dichlorophosphines and amines were used as such.

In a 25 mL two necked round bottom flask were added:
3 mL of 2-methyltetrahydrofuran
dichlorophosphine precursor (1.8 mmoles, 0.01 eq. with respect to methyl acrylate).

In a 50 mL three necked round bottom flask equipped with a magnetic stirring device were added:
1 mL of 2-methyltetrahydrofuran
3 equivalents with respect to the dichlorophosphine precursor of diisopropylamine (5.4 mmoles).

The dichlorophosphine solution was progressively added to the amine solution under stirring (1400 rpm) over 1 hour while keeping the temperature of the reaction medium below 40° C. (exothermic reaction). Upon dichlorophosphine addition to the amine solution, there was formation of a white precipitate corresponding to the insoluble ammonium chloride salt by-product (in the case of the invention diisopropylammonium chloride). The mixture was then allowed to stir at ambient temperature and the reaction progress was monitored thanks to ³¹P NMR. Formation of the intermediate chloro(diisopropylamino)phosphine was confirmed by ³¹P NMR (for example for the chlorophenyl(diisopropylamino)phosphine a singlet was observed at +132.5 ppm).

After chloroaminophosphine intermediate formation completion (which usually requires 1 h00 of stirring at room temperature after the dichlorophosphine addition), 1 equivalent of a second amine (1.8 mmoles) was added to the mixture at room temperature under stirring and the reaction mass was allowed to stir at room temperature for an additional hour.

After bis-aminophosphine completion, the mixture was then filtered via cannula into a 100 mL three necked round bottom flask equipped with a magnetic stirrer, a condenser, a heater and a temperature probe and containing 32 mL of melted tert-butanol (2:1 v/v with respect to methyl acrylate). The mixture was then allowed to stir at 60° C. Immediately, 15.95 mL of methyl acrylate (15.15 g, 0.176 mole, 1 eq.) was carefully added over 1 hour (exothermy) into the reactor and the reaction progress was monitored thanks to ¹H NMR. Reactions were pursued until advancement ceased or until 1 day of reaction. The conversion of methyl acrylate was then estimated by ¹H NMR thanks to the integration of the methylene protons of the products and the methylene proton in the starting methyl acrylate.

In order to confirm that the targeted catalysts were successfully synthesized, the crude reaction medium was analyzed using ³¹P NMR. Indeed, this parameter (³¹P NMR chemical shift) was characteristic for the synthetized aminophosphine and the area under the peak was proportional to the molar concentration of the aminophosphine in solution. The ³¹P NMR spectrum was recorded using a Bruker Avance 400 MHz spectrometer.

Additionally,
the NMR yield of the phosphine (%) which corresponds to the molar selectivity of the aminophosphine synthesis reaction deduced from the peak areas in the ³¹P NMR spectra recorded on the Me-THE solution before transfer into the dimerization reactor;
the maximum conversion during acrylate dimerization for some of the trials presented above in table 1, which corresponds to the maximal conversion rate of methyl acrylate measured from ¹H NMR;
   were measured.

The Ratio of t-BuOH:Acrylate in v:v (and in mol/mol) was also given.

For Inv 4.4, reaction was started with 0.5 mol % initial dichlorophenylphosphine loading followed by the addition of additional amount of methyl acrylate (0.5 eq. to reach 0.33 mol % initial dichlorophenylphosphine loading) after 20 h reaction time.

All the results are compiled in table 1 below:

TABLE 1
Aminophosphines screening study results - dimerization process

| | Chloro | | | | Chloro | NMR | | | |
|---|---|---|---|---|---|---|---|---|---|
| | Phos | | | | Phos | yield | | Ratio | mol/mol |
| | phine | | | ³¹P | phine | Phos | Max. | tBuOH: | (tBuOH/ |
| | pre | Amine | Amine | shift | loading | phine | Con- | Acryl- | MeAcryl- |
| Nº | cursor | 1 | 2 | (ppm) | (mol%) | (%) | version | ate v:v | ate) |
| Cp 1 | 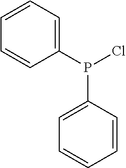 [SMILES: ClP(c1ccccc1)c1ccccc1] | 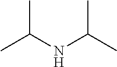 | — | 37.1 | 1 | 41 | 0 | 2:1 | 1.90 |
| Cp 2 | 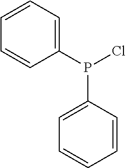 [SMILES: ClP(c1ccccc1)c1ccccc1] | 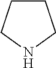 | — | 46.9 | 1 | 83 | 10 | 2:1 | 1.90 |
| Inv. 1 | 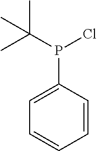 | 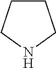 | -— | 65 | 0.5 | 79 | 73 | 2:1 | 1.90 |
| Inv. 2 | 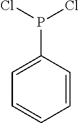 | 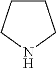 | 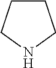 | 72.7 | 1 | 85 | 55 | 2:1 | 1.90 |
| Inv. 3.1 | 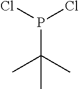 | 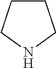 | 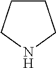 | 97.8 | 1 | 54 | 99 | 2:1 | 1.90 |
| Inv. 3.2 | 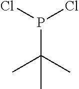 | 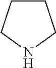 | 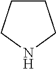 | 97.8 | 0.5 | 46 | 72 | 2:1 | 1.90 |
| Inv. 4.1 | 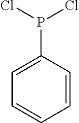 | 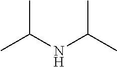 | 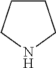 | 63.8 | 1 | 84 | 88 | 2:1 | 1.90 |
| Inv. 4.2 | 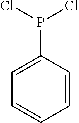 | 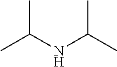 | 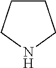 | 63.8 | 0.5 | 96 | 76 | 2:1 | 1.90 |
| Inv. 4.3 | 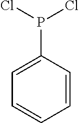 | 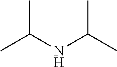 | 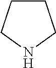 | 63.8 | 0.5 | 96 | 84 | 1:1 | 0.95 |
| Inv. 4.4 | 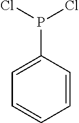 | 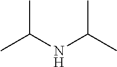 | 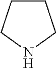 | 63.8 | 0.33 | 98 | 91 | 1:4 | 0.24 |
| Inv. 5 | 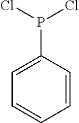 | 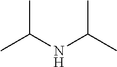 | 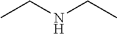 | 77.0 | 0.5 | 98 | — | 1:4 | 0.24 |
| Inv. 6 | 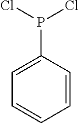 | 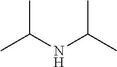 | 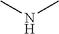 | 78.4 | 0.5 | 95 | 43 | 1:4 | 0.24 |
| Inv. 7 | 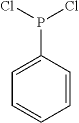 | 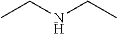 | 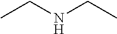 | 96.5 | 0.5 | 93 | 47 | 2:1 | 1.90 |

(Cp = Comparative Example)
All phosphines were synthesized.

Chlorodiphenylphosphine precursor gave only moderate yields of the aminophosphine by reaction with the diisopropylamine (Comp 1) and didn't afford a good catalytic activity. Chlorodiphenylphosphine precursor reacted with pyrrolidine (Comp 2) and also did not afford a good catalytic activity. On the other hand, the aminophosphines according to the invention (Inv 1 to 7) provided quite good catalytic activities.

The best system that displays the best performance was diisopropylamino-pyrrolidinophenylphosphine (Inv 4.1 to 4.4). It was very surprising to observe that at only 0.33 mol % of initial dichlorophosphine loading, an acrylate conversion of 91% was reached with the diisopropylamino-pyrrolidinophenylphosphine (Inv 4.4). In addition, it was observed that this phosphine was quite robust allowing an easier handling and even its recycling for several batches.

Presence of tert-butyl alcohol during the dimerization reaction allowed to improve the selectivity of the reaction toward the expected dimer. Surprisingly it was still possible to find suitable conditions allowing to use t-BuOH with very low amounts of basic aminophosphines without compromising the catalytic activity of the phosphine and providing good selectivity.

d) Optimization of the Dimerization Reaction: Bis(Diethylamino)Phenylphosphine Catalyzed (Initial 0.7 Mol % Dichlorophenylphosphine Precursor with Respect to Methyl Acrylate) Methyl Acrylate Dimerization in Tert-Butanol (1:4 v/v t-BuOH:Methyl Acrylate=0.24 Mol(t-BuOH)/Mol(Me-Acrylate)), 45° C.

All the reactions were conducted in carefully dried vessels and under an inert argon atmosphere. Methyl acrylate and tert-butanol were dried using 4 A molecular sieves and tert-butanol was distilled under argon prior to each reaction. Dichlorophenylphosphine and diethylamine were used as such.

In a 50 mL two necked round bottom flask were added:
15 mL of 2-methyltetrahydrofuran
4.2 mL of dichlorophenylphosphine (5.57 g, 0.031 mole, 0.007 eq.).

In a 100 mL three necked round bottom flask equipped with a magnetic stirring device are added:
20 mL of 2-methyltetrahydrofuran
12.9 mL of diethylamine (9.1 g, 0.124 mole, 0.028 eq.) (4 equivalents with respect to the dichlorophenylphosphine).

The dichlorophenylphosphine in 2-methyltetrahydrofuran solution was progressively added to the diethylamine solution under stirring (1400 rpm) over 1 hour while keeping the temperature of the reaction medium below 40° C. (exothermic reaction). Upon dichlorophenylphosphine addition, there was formation of a white precipitate corresponding to the ammonium chloride salt by-product (in this case diethylammonium chloride). At the end of the addition, the mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated via cannula into a 500 mL double jacketed reactor maintained at 45° C. equipped with a temperature probe, a condenser and a mechanical stirrer (propeller with four inclined plows) containing:
100 mL of distillated tert-butanol (1:4 v/v tert-butanol:methyl acrylate)
400 mL of methyl acrylate (380.8 g, 4.42 moles, 1 eq.)

The mixture was then allowed to stir at 45° C. during 19 hours. The reaction progress was monitored thanks to ¹H NMR. The conversion of methyl acrylate was estimated by ¹H NMR thanks to the integration of the methylene protons of the products and the methylene protons in the starting methyl acrylate. According to NMR, the conversion of the starting methyl acrylate was ˜92 mol %.

At the end of the reaction, the volatiles (t-BuOH, Me-THF and unconverted methyl acrylate) were distilled off allowing to recover 31 g of unreacted methyl acrylate. The desired product (dimethyl 2-methyleneglutarate) was then distilled under vacuum (160° C., 15 mbar) to afford 283 g of analytically pure product (isolated yield=75%). High boiling point by-products (methyl acrylate oligomers) that remained in the distillation vessel accounted for approximately 57 g (15%).

¹H NMR (CDCl₃, 400 MHz) δ (ppm): 6.03 (s, 1H), 5.46 (s, 1H), 3.60 (s, 3H), 3.51 (s, 3H), 2.48 (t, J=7.6 Hz, 2H), 2.37 (t, J=7.6 Hz, 2H).

¹³C NMR (CDCl₃, 101 MHz) δ (ppm): 172.73, 166.75, 138.76, 125.56, 51.59, 51.26, 32.66, and 27.17.

e) Bis(Diethylamino)Phenylphosphine Catalyzed (0.7 Mol % Dichlorophenylphosphine Precursor with Respect to Methyl Acrylate) Methyl Acrylate Dimerization in Tert-Butanol (1:8 v/v t-BuOH:Methyl Acrylate=0.12 Mol(t-BuOH)/Mol(Me-Acrylate)), 45° C.

All the reactions were conducted in carefully dried vessels and under an inert argon atmosphere. Methyl acrylate and tert-butanol were dried using 4 A molecular sieves and tert-butanol was distilled under argon prior to each reaction. Dichlorophenylphosphine and diethylamine were used as such.

In a 50 mL two necked round bottom flask are added:
15 mL of 2-methyltetrahydrofuran
4.2 mL of dichlorophenylphosphine (5.57 g, 0.031 mole, 0.007 eq.).

In a 100 mL three necked round bottom flask equipped with a magnetic stirring device were added:
20 mL of 2-methyltetrahydrofuran
12.9 mL of diethylamine (9.1 g, 0.124 mole, 0.028 eq.) (4 equivalents with respect to the dichlorophenylphosphine).

The dichlorophenylphosphine in 2-methyltetrahydrofuran solution was progressively added to the diethylamine solution under stirring (1400 rpm) over 1 hour while keeping the temperature of the reaction medium below 40° C. (exothermic reaction). Upon dichlorophenylphosphine addition, there was formation of a white precipitate corresponding to the ammonium chloride salt by-product (in this case diethylammonium chloride). At the end of the dichlorophosphine addition, the mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated via cannula into a 500 mL double jacketed reactor maintained at 45° C. equipped with a temperature probe, a condenser and a mechanical stirrer (propeller with four inclined plows) containing:
50 mL of distillated tert-butanol (1:8 v/v tert-butanol:methyl acrylate)
400 mL of methyl acrylate (380.8 g, 4.42 moles, 1 eq.).

The mixture was then allowed to stir at 45° C. during 19 hours. The reaction progress was monitored thanks to ¹H NMR. The conversion of methyl acrylate was estimated by ¹H NMR thanks to the integration of the methylene protons of the products and the methylene protons in the starting methyl acrylate. According to NMR, the conversion of starting methyl acrylate was ˜ 88 mol %. At the end of the reaction, the volatiles (t-BuOH, Me-THF and unconverted methyl acrylate) were distilled off allowing to recover 44 g of unreacted methyl acrylate.

The desired product (dimethyl 2-methyleneglutarate) was then distilled under vacuum (160° C., 15 mbar) to afford 271 g of analytically pure product (isolated yield=71%). High boiling point by-products (methyl acrylate oligomers) that remained in the distillation vessel accounted for 59 g (16%).

¹H NMR (CDCl₃, 400 MHz) δ (ppm): 6.03 (s, 1H), 5.46 (s, 1H), 3.60 (s, 3H), 3.51 (s, 3H), 2.48 (t, J=7.6 Hz, 2H), 2.37 (t, J=7.6 Hz, 2H).

¹³C NMR (CDCl₃, 101 MHz) δ (ppm): 172.73, 166.75, 138.76, 125.56, 51.59, 51.26, 32.66, and 27.17.

f) Bis(Diethylamino)Phenylphosphine Catalyzed (0.7 Mol % Dichlorophenylphosphine Precursor with Respect to Methyl Acrylate) Methyl Acrylate Dimerization in Tert-Butanol (1:8 v/v t-BuOH:Methyl Acrylate=0.12 Mol(t-BuOH)/Mol(Me-Acrylate)), 60° C., Average for 2 Batches.

All the reactions were conducted in carefully dried vessels and under an inert argon atmosphere. Methyl acrylate and tert-butanol were dried using 4 A molecular sieves and tert-butanol was distilled under argon prior to each reaction. Dichlorophenylphosphine and diethylamine were used as such.

In a 25 mL two necked round bottom flask were added:
8 mL of 2-methyltetrahydrofuran
2.1 mL of dichlorophenylphosphine (2.79 g, 0.0155 mole, 0.007 eq.).

In a 50 mL three necked round bottom flask equipped with a magnetic stirring device were added:
10 mL of 2-methyltetrahydrofuran
6.5 mL of diethylamine (4.6 g, 0.062 mole, 0.028 eq.) (4 equivalents with respect to the dichlorophenylphosphine).

The dichlorophenylphosphine in 2-methyltetrahydrofuran solution was progressively added to the diethylamine solution under stirring (1400 rpm) over 1 hour while keeping the temperature of the reaction medium below 40° C. (exothermic reaction). Upon dichlorophenylphosphine addition, there was formation of a white precipitate corresponding to the diethylammonium chloride salt by-product. The mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated via cannula into a 500 mL double jacketed reactor maintained at 60° C. equipped with a temperature probe, a condenser and a mechanical stirrer (propeller with four inclined plows) containing:
25 mL of distillated tert-butanol (1:8 v/v tert-butanol:methyl acrylate)
200 mL of methyl acrylate (190 g, 2.21 moles, 1 eq.).

The mixture was then allowed to stir at 60° C. during 20 hours. The reaction progress was monitored thanks to ¹H NMR. The conversion of methyl acrylate was estimated by ¹H NMR thanks to the integration of the methylene protons of the products and the methylene protons in the starting methyl acrylate. According to NMR, the conversion of starting methyl acrylate was ˜ 95 mol % (average 2 batches). At the end of the reaction, the volatiles (t-BuOH, Me-THF and unconverted methyl acrylate) were distilled off.

The desired product (dimethyl 2-methyleneglutarate) was then distilled under vacuum (140° C., 5 mbar) to afford 137 g of analytically pure product (average 2 batches, isolated yield=72%). High boiling point by-products (mainly methyl acrylate oligomers) that remained in the distillation vessel accounted for 40 g (21%, average 2 batches).

g) Bis(Diethylamino)Phenylphosphine Catalyzed (0.9 Mol % Dichlorophenylphosphine Precursor with Respect to Methyl Acrylate) Methyl Acrylate Dimerization in Tert-Butanol (1:4 v/v t-BuOH:Methyl Acrylate=0.24 Mol(t-BuOH)/Mol(Me-Acrylate)), 30° C.

All the reactions were conducted in carefully dried vessels and under an inert argon atmosphere. Methyl acrylate and tert-butanol were dried using 4 A molecular sieves and tert-butanol was distilled under argon prior to each reaction. Dichlorophenylphosphine and diethylamine were used as such.

In a 50 mL two necked round bottom flask are added:
20 mL of 2-methyltetrahydrofuran
5.43 mL of dichlorophenylphosphine (7.17 g, 0.04 mole, 0.009 eq.).

In a 100 mL three necked round bottom flask equipped with a magnetic stirring device are added:
20 mL of 2-methyltetrahydrofuran
16.6 mL of diethylamine (11.7 g, 0.16 mole, 0.036 eq.) (4 equivalents with respect to the dichlorophenylphosphine).

The dichlorophenylphosphine in 2-methyltetrahydrofuran solution was progressively added to the diethylamine solution under stirring (1400 rpm) over 1 hour while keeping the temperature of the reaction medium below 40° C. (exothermic reaction). Upon dichlorophenylphosphine addition, there was formation of a white precipitate corresponding to the diethylammonium chloride salt by-product. At the end of the dichlorophosphine addition, the mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated via cannula into a 500 mL double jacketed reactor maintained at 30° C. equipped with a temperature probe, a condenser and a mechanical stirrer (propeller with four inclined plows) containing:
100 mL of distillated tert-butanol (1:4 v/v tert-butanol:methyl acrylate)
400 mL of methyl acrylate (380.8 g, 4.42 moles, 1 eq.).

The mixture was then allowed to stir at 30° C. during 20 hours. The reaction progress was monitored thanks to ¹H NMR. At this stage the conversion level of methyl acrylate was 93% as estimated by ¹H NMR. The volatiles (2-methyltetrahydrofuran, t-BuOH and remaining methyl acrylate) were then removed under vacuum allowing to recover 27 g of methyl acrylate. Then, the desired dimethyl 2-methyleneglutarate was distilled off under vacuum (160° C., 15 mbar) in order to recover 284 g of analytically pure product corresponding to an isolated purified yield of 75%. High boiling point by-products (mainly methyl acrylate oligomers) that remained in the distillation vessel accounted for 49 g (13%).

h) Bis(Diethylamino)Phenylphosphine Catalyzed (0.9 Mol % Dichlorophenylphosphine Precursor with Respect to Methyl Acrylate) Methyl Acrylate Dimerization in Tert-Butanol (1:4 v/v t-BuOH:Methyl Acrylate=0.24 Mol(t-BuOH)/Mol(Me-Acrylate)), 30° C., Progressive Addition of Methyl Acrylate.

All the reactions were conducted in carefully dried vessels and under an inert argon atmosphere. Methyl acrylate, tert-butanol and diethylamine were dried using 4 A molecular sieves and tert-butanol was distilled under argon prior to each reaction. Dichlorophenylphosphine were used as such.

In a 50 mL two necked round bottom flask are added:
20 mL of 2-methyltetrahydrofuran
5.43 mL of dichlorophenylphosphine (7.17 g, 0.04 mole, 0.009 eq.).

In a 100 mL three necked round bottom flask equipped with a magnetic stirring device were added:
20 mL of 2-methyltetrahydrofuran
16.6 mL of diethylamine (11.7 g, 0.16 mole, 0.036 eq.) (4 equivalents with respect to the dichlorophenylphosphine).

The dichlorophenylphosphine in 2-methyltetrahydrofuran solution was progressively added to the diethylamine solution under stirring (1400 rpm) over 1 hour while keeping the temperature of the reaction medium below 40° C. (exothermic reaction). Upon dichlorophenylphosphine addition, there was formation of a white precipitate corresponding to the diethylammonium chloride salt by-product. At the end of the dichlorophosphine addition, the mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated via cannula into a 500 mL double jacketed reactor maintained at 30° C. equipped with a temperature probe, a condenser and a mechanical stirrer (propeller with four inclined plows) containing:
100 mL of distillated tert-butanol (1:4 v/v tert-butanol:methyl acrylate)
100 mL of methyl acrylate (95.2 g, 1.105 moles, 0.25 eq.).

Then 300 mL of methyl acrylate (285.6 g, 3.315 moles, 0.75 eq.) were progressively added over 4 hours into the reactor. At the end of the addition, the mixture was then allowed to stir at 30° C. during 16 hours. The reaction progress was monitored thanks to ¹H NMR. At this stage the conversion level of methyl acrylate was 92% as estimated by ¹H NMR.

The volatiles (2-methyltetrahydrofuran, t-BuOH and remaining methyl acrylate) were then removed under vacuum allowing to recover 29 g of methyl acrylate. Then, the desired dimethyl 2-methyleneglutarate was distilled off under vacuum (160° C., 15 mbar) in order to recover 288 g of analytically pure product corresponding to an isolated purified yield of 76%. High boiling point by-products (methyl acrylate oligomers) that remained in the distillation vessel accounted for 49 g (13%).

i) (Diisopropylamino)Pyrrolidinophenylphosphine Catalyzed (0.4 Mol % Dichlorophenylphosphine Precursor with Respect to Methyl Acrylate) Methyl Acrylate Dimerization in Tert-Butanol (1:8 v/v t-BuOH:Methyl Acrylate=0.12 Mol(t-BuOH)/Mol(Me-Acrylate)), 60° C. with Catalyst Recycling.

All the reactions were conducted in carefully dried vessels and under an inert argon atmosphere. Methyl acrylate and tert-butanol were dried using 4 A molecular sieves and tert-butanol was distilled under argon prior to each reaction. Dichlorophenylphosphine, diisopropylamine and pyrrolidine were used as such.

In a 50 mL two necked round bottom flask were added:
20 mL of 2-methyltetrahydrofuran
3.6 mL of dichlorophenylphosphine (4.77 g, 0.027 mole, 0.012 eq.).

In a 100 mL three necked round bottom flask equipped with a magnetic stirring device were added:
20 mL of 2-methyltetrahydrofuran
11.15 mL of diisopropylamine (8.05 g, 0.08 mole, 0.036 eq.) (3 equivalents with respect to the dichlorophenylphosphine).

The dichlorophenylphosphine in 2-methyltetrahydrofuran solution was progressively added to the diisopropylamine solution under stirring (1400 rpm) over 1 hour while keeping the temperature of the reaction medium below 40° C. (exothermic reaction). The mixture was allowed to stir at room temperature and 1 equivalent (0.027 mole, 1.92 g) of pyrrolidine was added to the reaction mixture which was allowed to stir at room temperature for an additional hour in order to complete bis-(amino)phosphine formation.

The reaction mixture was filtered via cannula into a 500 mL double jacketed reactor equipped with a temperature probe, a condenser and a mechanical stirrer (propeller with four inclined plows) containing:
25 mL of distillated tert-butanol (1:8 v/v tert-butanol:methyl acrylate)
200 mL of methyl acrylate (190.1 g, 2.2 moles, 1 eq.).

The mixture was then allowed to stir at 60° C. during 19 hours. The reaction progress was monitored thanks to ¹H NMR. According to ¹H NMR, the conversion of starting methyl acrylate was ˜ 86 mol %.

The volatiles (t-BuOH, Me-THF and unconverted methyl acrylate) are distilled off allowing to recover 20 g of methyl acrylate.

The desired product (dimethyl 2-methyleneglutarate) is then distilled under vacuum (125° C., 7 mbar) to afford 103 g of analytically pure product.

Then, 190 g of methyl acrylate (2.2 moles, 1 eq.) is added to the residue still containing active phosphine catalyst followed by the addition of 20 g of tert-butanol. The mixture is allowed to stir at 60° C. for an additional 16 h00 in order to convert a second batch of methyl acrylate. Volatiles are distilled off allowing to recover 37 g of methyl acrylate and the product is distilled under vacuum (125° C., 8 mbar) to afford 126 g of analytically pure product.

Finally, an additional 190 g of methyl acrylate (2.2 moles, 1 eq.) is added to the residue which still contains active phosphine and the mixture is stirred again at 70° C. during 20 hours. At the end of the reaction the volatiles are removed under vacuum to recover 44 g of methyl acrylate, and the product is distilled under vacuum to afford 91 g of pure product.

In total, 320 g of dimethyl 2-methyleneglutarate product is recovered corresponding to a global isolated purified yield of 56%.

This is the first example of an aminophosphine catalyst that can be recycled after acrylate dimerization reaction.

j) Influence of the Presence/Absence of t-BuOH

All the reactions were conducted in carefully dried vessels under an inert argon atmosphere. Methyl acrylate were dried using 4 A molecular sieves. Dichlorophosphines and amines were used as such.

In a 25 mL two necked round bottom flask were added:
18 mL of 2-methyltetrahydrofuran
dichlorophosphine precursor (10.5 mmoles, 0.005 eq. with respect to methyl acrylate).

In a 50 mL three necked round bottom flask equipped with a magnetic stirring device were added:
6 mL of 2-methyltetrahydrofuran
3 equivalents with respect to the dichlorophosphine precursor of diisopropylamine (31.4 mmoles).

The dichlorophosphine solution was progressively added to the amine solution under stirring (1400 rpm) over 1 hour while keeping the temperature of the reaction medium below 40° C. (exothermic reaction). Upon dichlorophosphine addition to the amine solution, there was formation of a white precipitate corresponding to the insoluble diisopropylammonium chloride salt by-product. The mixture was then allowed to stir at ambient temperature and the reaction progress was monitored thanks to ³¹P NMR. Formation of the intermediate chloro(diisopropylamino)phosphine was confirmed by ³¹P NMR (for example for the chlorophenyl(diisopropylamino)phosphine a singlet was observed at +132.5 ppm)

After chloroaminophosphine intermediate formation completion (which usually requires 1 h of stirring at room temperature after the dichlorophosphine addition), 1 equivalent of pyrrolidine (10.5 mmoles) was added to the mixture at room temperature under stirring and the reaction mass was allowed to stir at room temperature for an additional hour.

After bis-aminophosphine completion, the mixture was then filtered via cannula into a 500 mL double-jacketed reactor equipped with a mechanical stirrer (propeller with four inclined plows), a condenser, a heater and a temperature probe and containing 190 mL methyl acrylate (180 g, 2.1 moles). The mixture was then allowed to stir at 60° C. during 20 hours. The conversion of methyl acrylate was then estimated by ¹H NMR thanks to the integration of the methylene protons of the products and the methylene proton in the starting methyl acrylate. At 66% methyl acrylate conversion, the selectivity towards dimer as determined by 1H NMR was estimated to be 78 mol %.

In comparison when t-BuOH was present during the reaction (1:1 v/v tert-BuOH:methyl acrylate), 0.5 mol % dichlorophenylphosphine initial loading, 60° C. (corresponding to Inv 4.3), at similar conversion level (66%), the selectivity toward dimer was 87% demonstrating clearly the positive impact of t-BuOH on the reaction selectivity.

### k) Methyl Acrylate Dimerization Loading Impact

The reactions are conducted in carefully dry vessels and under an inert argon atmosphere.

Tert-butanol solvent has been flash distilled under argon prior to the reaction and anisole was dried over activated molecular sieve 4 A overnight prior to the reaction. The reactants diethylamine and methyl acrylate were also dried over activated molecular sieve 4 A overnight prior to the reaction.

K1) In a 250 mL double-jacketed reactor equipped with a temperature probe, a mechanical stirrer (propeller with 4 inclined plow) and baffles are added at room temperature:
40 g of anisole.
18.4 mL of diethylamine (12.98 g, 177 mmoles)

A dichlorophenylphosphine (DCPP) solution in anisole (previously prepared by diluting 6.17 mL of DCCP 97% purity (8.14 g, 44 mmoles) in 30 g of anisole) is progressively added under stirring (500 rpm) at room temperature to the diethylamine in anisole solution over 1 hour (exothermy).

Over the course of the addition, there is formation of a precipitate (NH₂Et₂Cl) resulting to a gel-like solution. At the end of the addition, an additional amount of 30 g of anisole is added to the mixture in order to reduce the viscosity of the suspension and the reaction medium is allowed to stir at room temperature during 1 h30. ³¹P NMR analysis confirms at this stage the full conversion of DCPP to the desired bis(diethylamino)phenylphosphine.

The suspension is then easily filtered in a filtration cell under argon using a 6 μm filter cloth. The solid is washed with an additional 60 g of anisole. Overall 138.4 g of a clear yellow solution is obtained as the filtrate. Quantitative ³¹P NMR analysis of the solution using triethyl phosphate as an internal probe allows determining the concentration of the aminophosphine catalyst in solution: 4.6 wt % corresponding to 6.3 g of catalyst (25 mmoles) which corresponds to a catalyst loading of only 0.28 mol % with respect to methyl acrylate.

In a 1.5 L double-jacketed reactor equipped with a condenser, a temperature probe, a mechanical stirrer (propeller with 4 inclined plows) and baffles is added 157.8 g of tert-butanol (200 mL) followed by the addition of the previously catalyst in anisole solution (138.4 g). The solution is then allowed to stir at 40° C. (500 rpm) and 760 g of methyl acrylate (8.828 moles) is progressively added into the solution over 4 h00 (exothermy). At the end of methyl acrylate addition the reaction mixture is stirred at 40° C. overnight and the reaction progress is followed thanks to quantitative GC chromatography.

The methyl acrylate conversion is followed up over time (as well as the dimer yield) and the kinetic curves are represented in the graphs below (left: methyl acrylate conversion over time; right: dimer yield over time). As it can be seen on the graph below (black curves) with only 0.28 mol % catalyst loading a final conversion of 66% is obtained after 24 h00 at 40° C. corresponding to a dimer yield of 58%.

K2) In a 250 mL double-jacketed reactor equipped with a temperature probe, a mechanical stirrer (propeller with 4 inclined plow) and baffles are added at room temperature:
40 g of anisole.
25.7 mL of diethylamine (18.17 g, 247 mmoles)

A dichlorophenylphosphine (DCPP) solution in anisole (previously prepared by diluting 8.44 mL of DCCP 99% purity (11.13 g, 62 mmoles) in 30 g of anisole) is progressively added under stirring (500 rpm) at room temperature to the diethylamine solution over 1 hour (exothermy).

Over the course of the addition, there is formation of a precipitate (NH₂Et₂Cl) resulting to a gel-like solution. At the end of the addition, the reaction medium is allowed to stir at 40° C. during 30 minutes. ³¹P NMR analysis confirms at this stage the full conversion of DCPP to the desired bis(diethylamino)phenylphosphine.

The suspension is then filtered via cannula into an intermediate flask and the solid is washed with an additional 90 g of anisole. Overall 153.7 g of a clear yellow solution is obtained. Quantitative ³¹P NMR analysis of the solution using triethyl phosphate as an internal probe allows determining the concentration of the aminophosphine catalyst in solution: 8.6 wt % corresponding to 13.2 g of catalyst (52 mmoles) which corresponds to a catalyst loading of only 0.6 mol % with respect to methyl acrylate.

In a 1.5 L double-jacketed reactor equipped with a condenser, a temperature probe, a mechanical stirrer (propeller with 4 inclined plows) and baffles is added 157.8 g of tert-butanol (200 mL) followed by the addition of the previously catalyst in anisole solution (153.7 g). The solution is then allowed to stir at 40° C. (500 rpm) and 760 g of methyl acrylate (8.828 moles) is progressively added into the solution over 4 h00. At the end of the methyl acrylate addition the reaction mixture is stirred at 40° C. overnight and the reaction progress is followed thanks to quantitative GC chromatography.

When following the methyl acrylate conversion over time (as well as dimer yield) and the kinetic curves, it can be seen that with only 0.6 mol % catalyst loading a final conversion >99% is obtained after 25 h00 at 40° C. corresponding to a dimer yield of 82%.

K3) In a 250 mL double-jacketed reactor equipped with a temperature probe, a mechanical stirrer (propeller with 4 inclined plow) and baffles are added at room temperature:
40 g of anisole.
25.7 mL of diethylamine (18.17 g, 247 mmoles)

A dichlorophenylphosphine (DCPP) solution in anisole (previously prepared by diluting 8.44 mL of DCCP 99% purity (11.13 g, 62 mmoles) in 30 g of anisole) is progressively added under stirring (500 rpm) at room temperature to the diethylamine solution over 1 hour (exothermy).

Over the course of the addition, there is formation of a precipitate (NH₂Et₂Cl) resulting to a gel-like solution. At the end of the addition, the reaction medium is allowed to stir at 40° C. during 30 minutes. ³¹P NMR analysis confirms at this stage the full conversion of DCPP to the desired bis(diethylamino)phenylphosphine.

The suspension is then filtered via cannula into an intermediate flask and the solid is washed with an additional 90 g of anisole. Overall 142.0 g of a clear yellow solution is obtained. Quantitative ³¹P NMR analysis of the solution using triethyl phosphate as an internal probe allows determining the concentration of the aminophosphine catalyst in solution: 7.2 wt % corresponding to 10.22 g of catalyst (41 mmoles) which corresponds to a catalyst loading of only 0.46 mol % with respect to methyl acrylate.

In a 1.5 L double-jacketed reactor equipped with a condenser, a temperature probe, a mechanical stirrer (propeller with 4 inclined plows) and baffles is added 157.8 g of tert-butanol (200 mL) followed by the addition of 760 g of methyl acrylate (8.828 moles). The previously prepared catalyst in anisole solution (142.0 g) is then added to the solution under stirring (500 rpm, exothermy). The solution is then allowed to stir at 40° C. overnight and the reaction progress is followed thanks to quantitative GC chromatography.

The methyl acrylate conversion is followed up over time (as well as dimer yield) and the kinetic curves are showing that with only 0.46 mol % catalyst loading a final conversion of 87% is obtained after 48 h00 at 40° C. corresponding to a dimer yield of 76%.

### 2. Dimethyl 2-Methyleneglutarate Catalytic Hydrogenation to Dimethyl 2-Methylglutarate

The substrate dimethyl 2-methyleneglutarate (50 g, 0.29 mole) obtained according to the dimerization reaction described above (Inv 4.4) was first added into a 100 mL autoclave reactor equipped with a mechanical stirrer (Rushton turbine) followed by the addition of the Pd/C (3%) catalyst (powder, 51% moisture content, 1 g wet corresponding to 0.49 g dry, 1 wt % with respect to the substrate). The reactor was then tightly sealed and was purged 3 times with 20 bar of nitrogen followed by 3 times with 5 bar of hydrogen. The reaction mixture was allowed to stir at 1400 rpm and the temperature of the reaction mixture was then set at 40° C. The reaction medium was then allowed to stir at 40° C., 5 bar hydrogen pressure (1400 rpm) during 6 hours and hydrogen consumption was followed over time.

At the end of the reaction which was confirmed by the absence of hydrogen consumption, the reaction mixture was allowed to cool down at room temperature, stirring was stopped and the autoclave was depressurized. The reactor was purged with nitrogen, the crude was removed from the reactor and the catalyst was removed through filtration. The product dimethyl 2-methylglutarate was obtained after catalyst filtration as a clear liquid (50 g corresponding to a yield of 99%) and was used as such.

3. Synthesis of 2-Methylenepentanedioic Acid from Dimethyl 2-Methylenepentanedioate

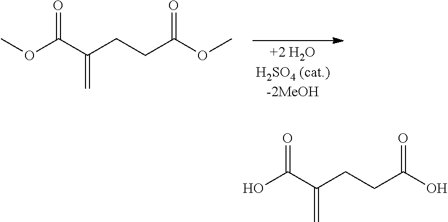

In a 2 L double-jacketed reactor equipped with a mechanical stirrer (propeller with four inclined plows), baffles, a temperature probe and a distillation column connected to a receiver are added:
700 g (4.07 mol, 1 eq.) of dimethyl 2-methylenepentanedioate.
879 mL of water (48.8 mol, 12 eq).
Sulfuric acid 95% (9 mL, 16.6 g, 0.163 mole, 4 mol % with respect to dimethyl 2-methylenepentanedioate) which is added drop-wise into the reaction mixture at room temperature thanks to an addition funnel.

The mixture is then stirred at 120° C. and the reaction progress is followed by 1H NMR analysis. Over the course of the reaction, generated methanol is distilled out from the reaction media in order to displace reaction equilibrium toward the desired methyleneglutaric acid.

After 2 h30 stirring at 120° C., ¹H NMR analysis indicates a slow conversion of the diester to the diacid, therefor additional amount of sulfuric acid is added into the reaction mixture to speed up the kinetics: 2.26 mL (0.04 mol, 1 mol %) and the temperature of the mixture is increased to 130° C.

However, after 2 h additional stirring at 130° C. the conversion of the diester is still too slow, therefor 2.26 mL (0.04 mol, 1 mol %) of H₂SO₄ is again added into the reaction mass.

After 11 h30 stirring at 130° C., 1060 mL of a water/MeOH mixture were distilled off and 50 mL of fresh water is added into the reaction vessel.

After 16 h30 stirring at 130° C., ¹H NMR analysis indicates around 10 mol % of remaining unhydrolyzed ester functions and significant formation of polymer by-products.

At this stage the recovered distillate mass is 1195 g containing 6 g of insoluble starting diester.

The temperature of the reaction medium is lowered to 80° C. and 36 mL of a 35 wt % NaOH aqueous solution (2 eq. with respect to H₂SO₄) is slowly added into the vessel to neutralize the catalyst (exothermy).

The reactor content maintained at 80° C. is drained into a beaker while constantly stirring and the mixture solidifies into an increasingly firm white paste on cooling. 147 mL of water are added to the paste in order to have a filterable liquid paste and the mixture is allowed to cool down at room temperature to complete precipitation of the diacid product.

The product is then filtered through a sintered filter and a very viscous filtrate is obtained.

The cake is washed 4 times with 80 mL of water then 6 times with 70 mL of water, shaking the mixture well before each filtration.

The resulting aqueous filtrate which has precipitated at room temperature overnight is filtered and rewashed 10 times with 20 mL of water to collect additional product.

The solid fractions are gathered and the product is dried under vacuum at 50° C. (10 mbar) for 2 hours to afford 286 g of a white powder with over 98 wt % organic purity and containing 3 wt % water corresponding to 48% isolated yield.

### NMR Spectra:

¹H NMR (MeOD-d₄, 400 MHz) δ (ppm): 6.16 (s, 1H), 5.63 (s, 1H), 2.6-2.56 (t, J=7.6 Hz, 2H), 2.51-2.47 (t, J=7.6 Hz, 2H).

¹³C NMR (MeOD-d₄, 101 MHz) δ (ppm): 176.7, 170.12, 141.16, 126.38, 34.07, 28.52.

1. A process for producing a dimer according to formula (II) comprising a step i) of dimerization of alkyl acrylates according to formula (I) to obtain a dimer according to formula (II) using a catalyst according to formula (III), according to the following reaction scheme: 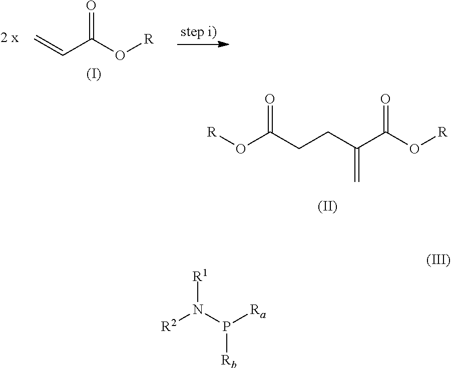
wherein
R is an alkyl group;
R₁ and R₂ which are identical or different, are either aliphatic groups or form together with the N atom a heteroaliphatic cycle;
Rₐ is a hydrocarbyl group;
R_{b} is either an aliphatic group or NR₃R₄ with R₃ and R₄ being identical or different, and being either aliphatic groups or forming together with the N atom a heteroaliphatic cycle;
and wherein said step i) of dimerization is performed in presence of a compound A being a tertiary alcohol or a silanol.

2. The process according to claim 1, wherein compound A is a tertiary alcohol.

3. The process according to claim 1, wherein the molar ratio [compound A]/[alkyl acrylate according to formula (I)] is selected from about 4:1 to about 0.01:1.

4. The process according to claim 1, wherein R is a C₁-C₁₈.

5. The process according to claim 1, wherein R₁ and R₂ are identical linear or branched alkyl groups comprising from 1 to 6 carbon atoms.

6. The process according to claim 1, wherein R₁ and R₂ form together with the N atom a heteroaliphatic cycle comprising from 3 to 5 carbon atoms.

7. The process according to claim 1, wherein Rₐ is either an aromatic or an aliphatic group.

8. The process according to claim 1, wherein R_{b} is NR₃R₄ with R₃ and R₄ being identical or different, and being either an aliphatic group or forming together with the N atom a heteroaliphatic cycle.

9. The process according to claim 1, wherein Rₐ is a phenyl, R₁ and R₂ are ethyl and R_{b} is NR₃R₄ with R₃ and R₄ being ethyl.

10. The process according to of claim 1, wherein the step i) of dimerization is performed in an organic solvent.

11. The process according to claim 1, wherein the dimerization step i) is performed at a temperature ranging from about 20° C. to about 120° C.

12. The process according to claim 1, further comprising an initial step 0) of preparation of the catalyst according to formula (III) by reacting a compound according to formula (IV) 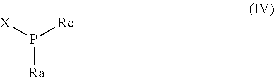
wherein
X is a chloride, a bromide or an iodide;
Rₐ is as defined in claim 1;
R_{c} is either X or R_{b}, wherein R_{b} is as defined in claim 1;
with
an amine of formula V: R₁R₂NH (V), with R₁ and R₂ being as defined in claim 1, when R_{c} is R_{b} or
both an amine of formula (V) and an amine of formula (V′): R₃R₄NH (V′), with R₃ and R₄ being as defined in claim 1 when R_{c} is X.

13. The process according to claim 12, wherein step 0) and step i) are consecutive steps performed without isolation of the catalyst after step 0).

14. The process for producing a compound according to formula (VI), comprising the process as defined in claim 1, followed by a step ii) of hydrogenation of the dimer according to formula (II) obtained in the step of dimerization using H₂ and a hydrogenation catalyst to obtain a compound of formula (VI) 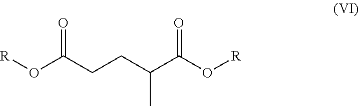
wherein R is as defined in claim 1.

15. The process for producing a compound according to formula (VII), comprising the process as defined in claim 1, followed by a step ii′) of hydrolysis of the dimer according to formula (II) obtained in the step of dimerization using acid catalysts to obtain a compound of formula (VII) [chemical structure]
